(19) United States
(12) Patent Application Publication
(10) Pub. No.: US 2026/0011004 A1
(43) Pub. Date: Jan. 8, 2026

(54) METHOD AND DEVICE FOR PREDICTING DISEASE THROUGH WRINKLE DETECTION

(71) Applicant: LULULAB INC., Seoul (KR)

(72) Inventors: Huisu YOON, Seoul (KR); Semin KIM, Ansan-si, Gyeonggi-do (KR); Jong Ha LEE, Hwaseong-si, Gyeonggi-do (KR); Sangwook YOO, Seoul (KR); Yongjoon CHOE, Seoul (KR)

(21) Appl. No.: 18/879,935

(22) PCT Filed: Jun. 28, 2023
(86) PCT No.: PCT/KR2023/009028

§ 371 Date: Dec. 30, 2024

(30) Foreign Application Priority Data
Jul. 1, 2022 (KR) 10-2022-0081116

(51) Int. Cl. G06T 7/00; G16H 50/20; H04N 23/667

(52) U.S. Cl. CPC G06T 7/0012; G16H 50/20; H04N 23/667; G06T 2207/20081; G06T 2207/20084; G06T 2207/30088

(57) ABSTRACT

A server for diagnosing a disease of a user through wrinkle detection includes an interface unit for displaying an interface for selecting a photographing objective through a display linked with the server, and acquiring, from the user, an input signal indicating the photographing objective, a photography unit for controlling a skin photography device to capture an image of the skin, an image analysis unit for acquiring the image of the skin from the skin photography device, performing preprocessing on the basis of landmarks based on the image of the skin and determining the skin type and skin characteristics of the user based on the preprocessed image, a disease analysis unit for detecting wrinkles of the user based on the image of the skin, and diagnosing a disease of the user based on the detected wrinkles and a solution provision unit for providing a customized solution according to the diagnosed disease.

### TECHNICAL FIELD

The present disclosure relates to a method and a device for predicting a disease through wrinkle detection and, more particularly, to a method and a device for detecting wrinkles of a user on the basis of an artificial neural network and diagnosing the disease of the user on the basis of the detected wrinkles of the user.

### BACKGROUND ART

Unless otherwise indicated herein, the matters described in this section are not prior art to the claims of this application and their inclusion in this section is not an admission that they are prior art.

Recently, the risk of developing diseases has increased significantly among modern people due to increased consumption of unhealthy instant foods or fast foods, lack of activity, excessive work, etc. In particular, cardiovascular diseases such as hypertension, ischemic heart disease, coronary artery disease, arteriosclerosis, and stroke are rapidly increasing, and many other diseases, including pulmonary disease and liver disease, are also occurring.

For example, cardiovascular disease is difficult to predict because it presents with symptoms such as nosebleeds, headaches, and dizziness, and if left untreated, it may lead to complex surgical procedures or, in severe cases, death. However, most modern people are reluctant to visit a hospital with only mild symptoms due to concerns about time and cost, and in fact, symptoms of cardiovascular disease may be easily encountered in daily life and are often overlooked, making it difficult to recognize whether or not cardiovascular disease has developed unless a professional examination is performed.

Therefore, there is a need to develop a method that allows users to more conveniently identify their symptoms when mild symptoms occur. Recently, many technologies for analyzing the skin of a user through the facial or body image of a user have been developed, and accordingly, many methods for users to check their skin condition more easily are being used. On the basis of this, it is necessary to devise a method to provide improved medical services by using images captured through a device that photographs the skin to not only analyze the skin, but also diagnose the disease of a user, proactively notify the user of symptoms before the symptoms worsen, and provide a solution based on the result of the diagnosis.

### DISCLOSURE

### Technical Problem

In order to solve the above problems, the objective of the present disclosure is to provide a method and a device for detecting wrinkles of a user on the basis of an artificial neural network and diagnosing the disease of the user on the basis of the detected wrinkles of the user.

### Technical Solution

According to various embodiments, a server for diagnosing a disease of a user through wrinkle detection includes: an interface control unit which displays an interface for selecting a photographing objective through a display linked with the server, and which acquires, from the user, an input signal indicating the photographing objective; a photography control unit, which controls a skin photography device so as to capture an image of the skin of the user; an image analysis unit for acquiring the image of the skin of the user from the skin photography device, performing preprocessing on the basis of landmarks based on the image of the skin, and determining a skin type and a skin characteristic of the user on the basis of the preprocessed image of the skin; a disease analysis unit for detecting wrinkles of the user on the basis of the image of the skin, and diagnosing a disease of the user on the basis of the detected wrinkles of the user; and a solution provision unit for providing a customized solution according to the diagnosed disease of the user.

The photography control unit may photograph a user in a first photography mode through the skin photography device when obtaining an input signal indicating disease analysis, and photograph the user in a second photography mode through the skin photography device when obtaining an input signal indicating skin analysis, and the disease analysis unit may include a wrinkle detection model that is trained by using of a training input value training data consisting corresponding to a skin image of each of multiple users obtained from multiple user terminals, and a training output value corresponding to a wrinkle image or a wrinkle probability map of the user, and generates the wrinkle probability map corresponding to the user on the basis of a deep learning network consisting of a plurality of hidden layers, may input the preprocessed skin image of the user into the wrinkle detection model based on a convolutional neural network (CNN), and generate a wrinkle image or a wrinkle probability map corresponding to the skin image on the basis of an output of the wrinkle detection model, and

may detect wrinkles of the user on the basis of the wrinkle image or the wrinkle probability map, which is generated.

The disease analysis unit may determine a wrinkle occurrence region of a user, and determines whether the user has a disease on the basis of the wrinkle occurrence region and a degree of wrinkles occurring in the region.

The disease analysis unit may calculate a disease risk on the basis of age of the user, the wrinkle occurrence region, and the degree of the wrinkle, and set a weight for the degree of the wrinkle to be low as the age of the user increases, and set a weight for the degree of the wrinkle to be high as the age decreases on the basis of the obtained age information.

The solution provision unit may provide a medical diagnosis service or information on recommended lifestyle habits and recommended eating habits on the basis of the disease risk.

### Advantageous Effects

According to various embodiments disclosed in this document, it is possible to diagnose the inside of the body of a user through the external body images of the user, thereby providing convenience to the user.

Additionally, according to various embodiments, it is possible to provide an improved medical service by proactively informing a user of information about related disease when symptoms are mild.

In addition, according to various embodiments, it is possible to diagnose the disease of a user more accurately through relative analysis that takes into account the age of the user rather than absolutely analyzing the condition of the skin (wrinkle) simply.

In addition, various effects that are directly or indirectly identified through this document may be provided.

### DESCRIPTION OF DRAWINGS

FIG. 1 is a view illustrating a wrinkle-based disease diagnosis system according to an embodiment.

FIG. 2 is a diagram illustrating the main components of a wrinkle-based disease diagnosis server.

FIG. 3 is a view illustrating the process of detecting the wrinkles of a user through the skin image of the user.

FIG. 4 is a flowchart for performing simple skin analysis or disease analysis of a user depending on a photographing objective.

FIG. 5 is a flowchart for analyzing the disease risk of a user on the basis of age information of the user and wrinkle information of the user.

FIG. 6 is a diagram illustrating the configuration of the hardware of the wrinkle-based disease diagnosis server according to FIG. 1.

### MODE FOR INVENTION

The present disclosure may have various modifications and embodiments, and specific embodiments are illustrated in the drawings and described in detail in the detailed description. However, this is not intended to limit the present disclosure to a specific embodiment, but should be understood to include all modifications, equivalents or substitutes included in the spirit and technical scope of the present disclosure. In describing each drawing, similar reference numerals are used to refer to similar components.

Terms such as “first”, “second”, “A”, “B”, etc. may be used to describe various components, but such components should not be limited by such terms. The terms are used solely to distinguish one component from another. For example, without exceeding the scope of the present disclosure, the first component may be named the second component, and similarly, the second component may be named the first component. The term “and/or” includes any combination of a plurality of related described items or any one of a plurality of related described items.

When it is mentioned that a component is “connected” or “coupled” to another component, it should be understood that the component may be directly connected or coupled to the another component, but there may be other components present therebetween. On the other hand, when it is mentioned that a component is “directly connected” or “directly coupled” to another component, it should be understood that there are no other components therebetween.

The terminology used in this application is for the purpose of describing particular embodiments only and is not intended to limit the present disclosure. Singular expressions include plural expressions unless the context clearly indicates otherwise. In this application, it should be understood that terms such as “include” or “have” are intended to specify the presence of a feature, number, step, operation, component, part or combination thereof described in the specification, but do not exclude in advance the possibility of the presence or addition of one or more other features, numbers, steps, operations, components, parts or combinations thereof.

Unless otherwise defined, all terms used herein, including technical or scientific terms, have the same meanings as commonly understood by those skilled in the art to which the present disclosure belongs. Terms defined in commonly used dictionaries should be interpreted as having meanings consistent with their meanings in the context of the relevant art, and should not be interpreted in idealized or overly formal sense unless expressly defined in this application.

Hereinafter, preferred embodiments according to the present disclosure will be described in detail with reference to the attached drawings.

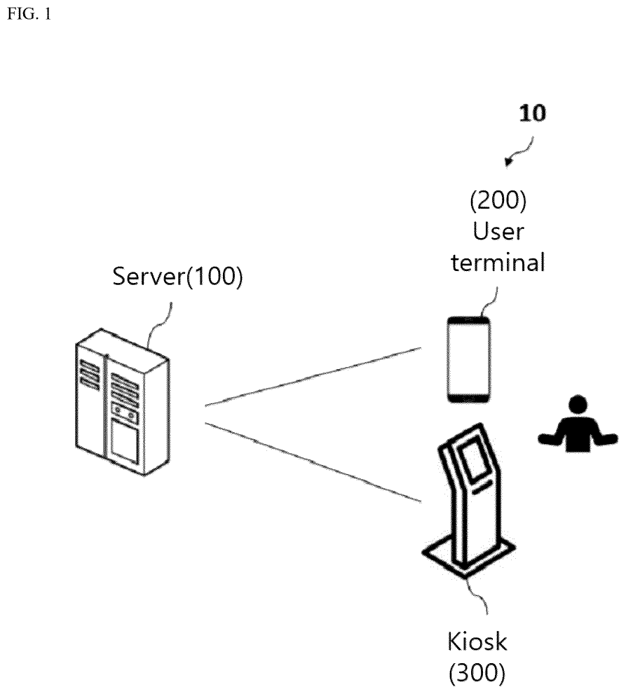

FIG. 1 is a view illustrating a wrinkle-based disease diagnosis system 10 according to an embodiment.

Referring to FIG. 1, the wrinkle-based disease diagnosis system 10 may include a wrinkle-based disease diagnosis server 100, a user terminal 200, a kiosk 300, etc. Operations described below may be performed or implemented through a platform (e.g., a web page and/or an application) controlled by the wrinkle-based disease diagnosis server 100 100. In other words, the wrinkle-based disease diagnosis server 100 may provide a website in which a user can input, register, and output various information by accessing the wrinkle-based disease diagnosis server 100 through a network by using the user terminal 200 and/or the kiosk 300, and may provide an application installed and executed in the user terminal 200 and/or the kiosk 300 so that various information is input, registered, and output.

The wrinkle-based disease diagnosis server 100 may take a picture of the skin of a user through a skin photography device (e.g., a camera of the user terminal 200, a camera of the kiosk 300, and/or other photographing device) linked to the wrinkle-based disease diagnosis server 100, and may perform simple skin analysis or disease analysis on the basis of the photographed skin of the user. For example, the wrinkle-based disease diagnosis server 100 may diagnose the skin type and/or skin characteristics of the user on the basis of the photographed skin of the user, or detect wrinkles of the user on the basis of the photographed skin of the user, and may diagnose or predict the disease of the user on the basis of the detected wrinkles of the user.

The user terminal 200 and/or the kiosk 300 may be a desktop computer, a laptop computer, a notebook, a smart phone, a tablet PC, a mobile phone, a smart watch, a smart glass, an e-book reader, a portable multimedia player (PMP), a portable game console, a navigation device, a digital camera, a digital multimedia broadcasting (DMB) player, a digital audio recorder, a digital audio player, a digital video recorder, a digital video player, or a personal digital assistant (PDA), etc. which are capable of communication.

The wrinkle-based disease diagnosis server 100, the user terminal 200, and the kiosk 300 may be each connected to a communication network to transmit and receive data between each other through the communication network. For example, as the communication network, various types of wired or wireless communication networks such as a local area network (LAN), a metropolitan area network (MAN), a global system for a mobile network (GSM), an enhanced data GSM environment (EDGE), high speed downlink packet access (HSDPA), wideband code division multiple access (W-CDMA), code division multiple access (CDMA), time division multiple access (TDMA), Bluetooth, Zigbee, Wi-Fi, voice over Internet protocol (VOIP), LTE Advanced, IEEE802.16m, WirelessMAN-Advanced, HSPA+, 3GPP long term evolution (LTE), Mobile WiMAX (IEEE 802.16e), UMB (formerly EV)-DO Rev. C), Flash-OFDM, iBurst and MBWA (IEEE 802.20) systems, HIPERMAN, beam-division multiple access (BDMA), world interoperability for microwave access (Wi-MAX), and 5G may be used.

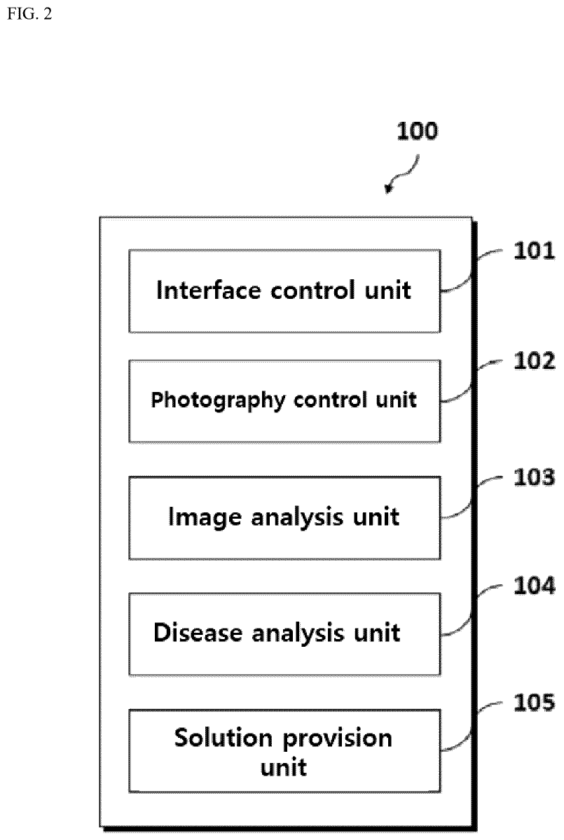

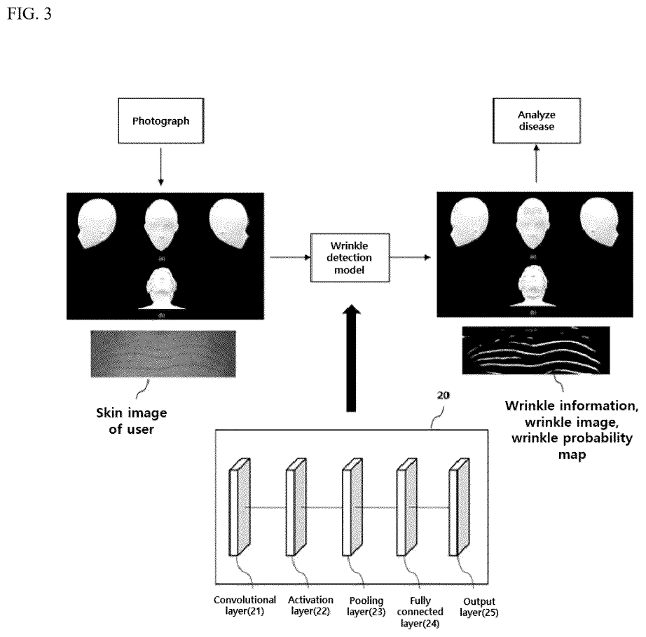

FIG. 2 is a diagram illustrating the main components of the wrinkle-based disease diagnosis server 100. FIG. 3 is a view illustrating the process of detecting the wrinkles of a user through the skin image of the user. The wrinkle-based disease diagnosis server 100 may include an interface control unit 101, a photography control unit 102, an image analysis unit 103, a disease analysis unit 104, and a solution provision unit 105.

The interface control unit 101 may display an interface for selecting a photographing objective through a display linked to the interface control unit 101 (e.g., a display of the user terminal 200 and/or a display of the kiosk 300). The interface control unit 101 may obtain an input signal indicating the photographing objective from a user. For example, the interface control unit 101 may obtain an input signal indicating that the photographing objective is skin analysis, may obtain an input signal indicating that the photographing objective is disease prediction, or may obtain an input signal indicating that the photographing objective is both skin analysis and disease prediction.

The photography control unit 102 may provide a photographing signal to the skin photography device (e.g., a camera of the user terminal 200, a camera of the kiosk 300, and/or other photographing devices) linked to the photography control unit 102.

When the photography control unit 102 obtains an input signal indicating disease analysis through the interface control unit 101, the photography control unit 102 may photograph a user in a first photography mode through the linked skin photography device. When the photography control unit 102 obtains an input signal indicating skin analysis through the interface control unit 101, the photography control unit 102 may photograph the user in a second photography mode through the linked skin photography device. The first photography mode may refer to a mode for photographing a facial region of a user, and the second photography mode may refer to a mode for photographing the ears and neck of the user in addition to the facial region of the user. The facial region may refer to the front surface of the head including the eyes, nose, and mouth of a user. The photography control unit 102 may obtain the skin image of a user from the skin photography device.

The image analysis unit 103 may perform preprocessing on the basis of landmarks based on the skin image of a user. The preprocessing may include processes before the skin image is subjected to skin analysis and/or disease analysis on the basis of artificial intelligence.

The image analysis unit 103 may determine whether the obtained skin image is an image suitable for training artificial neural network models or for skin analysis and/or wrinkle detection. The image analysis unit 103 may determine whether the obtained skin image is the suitable image on the basis of a brightness value thereof, a contrast value thereof, etc. In addition, the image analysis unit 103 may determine the obtained skin image to be an unsuitable image when the number of hairs that may interfere with wrinkle detection in the obtained skin image exceeds a preset number or the resolution of the skin image is lower than a preset resolution (e.g., 320×320).

The image analysis unit 103 may determine the skin type and/or skin characteristics of a user on the basis of the skin image of the user (or, referred to as a facial image). For example, skin characteristics may include skin moisture, oil, sebum, pH, sensitivity, elasticity, skin color/tone, pore condition, pigmentation, keratin condition, etc. The image analysis unit 103 may determine the skin characteristics of a user on the basis of a skin image captured and acquired through a separate measuring device (e.g., a measuring mask) other than the user terminal 200 and/or the kiosk 300. The image analysis unit 103 may determine the skin characteristics of a user, such as moisture, oil, sebum, pH, sensitivity, skin tone, pore condition, pigmentation, and dead skin cell condition, on the basis of the skin image, and may determine the skin type of the user on the basis of the determined skin characteristics.

The disease analysis unit 104 may detect wrinkles of a user on the basis of the obtained skin image of the user, unlike the operation of the image analysis unit 103 that determines the skin type or skin characteristics of a user. The disease analysis unit 104 may predict, determine, and/or diagnose a disease of the user on the basis of the detected wrinkles of the user.

The disease analysis unit 104 may input the skin image of a user, which has been preprocessed through the image analysis unit 103, into an artificial neural network model (e.g., a wrinkle detection model). In addition, the disease analysis unit 104 may train the artificial neural network model (e.g., the wrinkle detection model) by using multiple skin images obtained from multiple user terminals as training data. The disease analysis unit 104 may extract a wrinkle image or a wrinkle probability map corresponding to the skin image as an output of the artificial neural network model. The disease analysis unit 104 may binarize the extracted wrinkle image or the wrinkle probability map. The disease analysis unit 104 may detect the wrinkles of a user on the basis of the wrinkle image or the wrinkle probability map, which has been binarized.

Specifically, the image analysis unit 103 may apply a ROI mask to a region expected to be a wrinkle cluster and apply contrast-limited adaptive histogram equalization. The disease analysis unit 104 may then extract a wrinkle image or a wrinkle probability map from a preprocessed skin image by using the difference of Gaussian. Wrinkles may be segmented from a feature map by using an adaptive threshold method. The segmented wrinkles may be classified into wrinkles longer than a preset length, wrinkles shorter than a preset length, wrinkles deeper than a preset depth, and bifurcated wrinkles.

According to an embodiment, the wrinkle detection model based on an artificial neural network may be configured as a convolutional neural network 20. The convolutional neural network may include a convolutional layer 21 that receives image frames of a preset size as input images and extracts a feature map, an activation layer 22 that uses an activation function to determine whether to activate the output on the basis of extracted features, a pooling layer 23 that samples the output according to the activation layer 22, a fully connected layer 24 that performs classification by class, and an output layer 25 that finally outputs an output according to the fully connected layer 24.

The convolutional layer 21 may be a layer that extracts a feature of input data by convolving an input image and a filter with each other. Here, the filter, which is a function that detects a characteristic part of the input image, is usually expressed as a matrix and may be a function that is determined by continuous learning through training data. The feature extracted by the convolutional layer 21 may be referred to as a feature map. Additionally, an interval value for performing convolution may be referred to as stride, and a feature maps of a different size may be extracted depending on a value of the stride. In this case, when the size of the filter is smaller than the input image, the feature map has a smaller size than the original input image, and a padding process may be additionally performed to prevent a feature from being lost through multiple steps. In this case, the padding process may be a process of keeping the size of the input image and the size of the feature map equal by adding a preset value (e.g. 0 or 1) to the periphery of the generated feature map.

Here, the convolutional layer 21 according to an embodiment of the present disclosure may use a structure in which a 1×1 convolutional layer and a 3×3 convolutional layer are sequentially and repeatedly connected, but is not limited thereto.

The activation layer 22 is a layer that changes an extracted feature with value (or matrix) into a nonlinear value according to the activation function to determine whether to perform activation. As the activation function, a sigmoid function, a ReLU function, a softmax function, etc. may be used. For example, the softmax function may be a function that normalizes all input values to values between 0 and 1 and has the characteristic that the sum of output values is constantly 1.

The pooling layer 23 is a layer that selects a feature representing a feature map by performing subsampling or pooling on the output of the activation layer 22. Max pooling, which extracts a largest value for a certain region of the feature map, and average pooling, which extracts an average value for the certain region, may be performed. In this case, the pooling layer is not necessarily performed after the activation function but may be performed optionally.

Additionally, the convolutional neural network 20 may include a plurality of connection structures of the convolutional layer 21, the activation layer 22, and the pooling layer 23. For example, the convolutional neural network 20 may be a CNN-based shallow convolutional neural network (S-CNN), You Look Only Once (YOLO), Single Shot MultiBox Detector (SSD), Faster R-CNN, ResNet, U-Net, etc., or a deep neural network improved on the basis of these, but is not limited thereto. For example, the convolutional neural network 20 as a U-Net structure may include the plurality of connection structures of the convolutional layer 21, the activation layer 22, and the pooling layer 23, and may include at least one down-sampling process through down-sampling or max-pooling and at least one up-sampling process through up-convolution, and may perform a segmentation task on the basis of the above processes. This allows output data to have the same size as an input image, which is an original image, and allows a detected wrinkle image or wrinkle probability map to be superimposed on the input image.

The disease analysis unit 104 may determine or predict the current disease and/or potential future disease of a user on the basis of the detected wrinkles of a user. The disease analysis unit 104 may determine the occurrence region of the wrinkles of the user and calculate the degree of the wrinkles of a user at that region. The disease analysis unit 104 may calculate the wrinkle degree on the basis of the number of the detected wrinkles, the area of each of the detected wrinkles, and spacing (density) between the detected wrinkles. The disease analysis unit 104 may calculate the number of pixels occupied by each of the detected wrinkles and calculate the area of each of the wrinkles by calculating the sum of the calculated numbers of pixels. The disease analysis unit 104 may determine a wrinkle region including the detected wrinkles, and calculate a density between the wrinkles on the basis of the number of the wrinkles and the area of each of the wrinkles within the determined wrinkle region.

The disease analysis unit 104 may determine a disease of a user on the basis of the wrinkle occurrence region of the user and/or the degree of wrinkles of the user, and calculate a disease risk for the disease. The disease analysis unit 104 may calculate the disease risk on the basis of the wrinkle occurrence region, the wrinkle degree, and age of a user corresponding to the obtained age information of the user. The disease analysis unit 104 may set a weight for the wrinkle degree to be low as the age of the user increases and may set a weight for the wrinkle degree to be high as the age of the user decreases on the basis of the obtained age information. That is, as the age increases, the likelihood of the occurrence of wrinkles due to natural aging increases, so the disease risk may be calculated by taking this into account.

The disease analysis unit 104 may calculate the disease risk on the basis of susceptibility to wrinkle occurrence in each region in addition to the consideration of the age of a user. The susceptibility to wrinkle occurrence may be an indicator of whether each region is more or less prone to developing wrinkles. For example, since regions such as the forehead, a region around the eyes, and a region around the mouth are regions in which wrinkles are more likely to form due to changes in facial expressions, head movements, etc., the disease analysis unit 104 may determine susceptibility to wrinkle occurrence for the forehead, the region around the eyes, a region around the neck, and the region around the mouth to be high, determining weights for these regions to be low when calculating the disease risk, and may determine susceptibility to wrinkle occurrence for the chin, cheeks, earlobes, and nose to be low, setting weights for the regions to be high when calculating the disease risk.

The disease analysis unit 104 may determine the susceptibility to wrinkle occurrence on the basis of a facial image of each of multiple users, and calculate an average of the degrees of wrinkles for respective regions for the multiple users. The susceptibility to wrinkle occurrence may be determined to be close to 1 as the average of the degrees of the wrinkles for the regions increases, and the susceptibility to wrinkle occurrence may be determined to be close to 0 as the average of the degrees of the wrinkles for the regions decreases.

The disease analysis unit 104 may calculate the disease risk by using the following Mathematical expression 1.

$$G = \frac{d \times w}{\sqrt{a}} \qquad [ Mathematical expression 1 ]$$

$$w = {e}^{- ( \frac{{( x - {x}_{o} )}^{2}}{σ} )} , \qquad x > {x}_{o}$$

In Mathematical expression 1 above, G represents the disease risk, d represents the degree of wrinkles, W represents a weight for the degree of wrinkles, a represents the susceptibility to wrinkle occurrence in the region of wrinkled occurrence, x represents the age of a user, x₀ represents a preset reference age, and σ may be a parameter that controls change in weight (the slope of a weight graph). In other words, w may be a value that decreases with age. The parameter may be determined by gender, race, etc.

When a calculated disease risk exceeds a preset first threshold, the disease analysis unit 104 may determine that there is a disease related to a corresponding portion. For example, the disease analysis unit 104 may determine that wrinkles on earlobe are related to cerebrovascular disease, dementia, etc. When the disease risk based on the wrinkles on earlobe exceeds the first threshold, the disease analysis unit 104 may determine that there is a disease related to cerebrovascular disease or dementia. For example, the disease analysis unit 104 may determine that the forehead is associated with cardiovascular disease, etc., and may determine that cardiovascular disease is present when the disease risk based on forehead wrinkles exceeds the first threshold.

The solution provision unit 105 may recommend a medical diagnosis when the calculated disease risk exceeds a preset second threshold that is higher than the first threshold. The disease analysis unit 104 may provide a user with information on recommended lifestyle habits and/or information on recommended eating habits when the disease risk exceeds the first threshold but is lower than or equal to the second threshold.

The solution provision unit 105 may perform regular photographing through the photography control unit 102 and calculate a change in the wrinkle degree of an individual user. The solution provision unit 105 may list the wrinkle image or wrinkle probability map of the user according to a preset condition (e.g., order of disease risk or time order) based on the change of the wrinkle degree of the user, and provide the listed wrinkle image or wrinkle probability map to the user through the user terminal 200 and/or the kiosk 300. The solution provision unit 105 may determine a photographing cycle according to the calculated disease risk, and may determine the photographing cycle to be short as the disease risk increases, and may determine the photographing cycle to be long as the disease risk decreases.

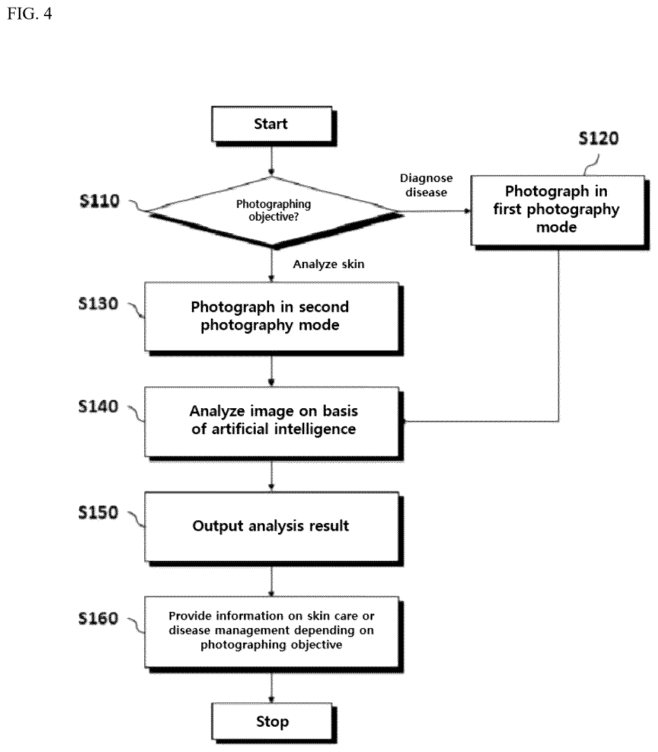

FIG. 4 is a flowchart for performing simple skin analysis or disease analysis of a user depending on a photographing objective.

The interface control unit 101 may obtain an input signal, from a user, indicating the photographing objective through the user terminal 200 (e.g., the display of the user terminal 200) or the kiosk 300 (e.g., the display of the kiosk 300). The photography control unit 102 may determine the photographing objective corresponding to the input signal in S110.

When the photography control unit 102 obtains an input signal indicating that the photographing objective is disease analysis, the photography control unit 102 may control the skin photography device (e.g., a camera of the user terminal 200, a camera of the kiosk 300, and/or other photographing devices) to photograph a user in the first photography mode in S120.

When the photography control unit 102 obtains an input signal indicating that the photographing objective is skin analysis, the photography control unit 102 may control the skin photography device (e.g., a camera of the user terminal 200, a camera of the kiosk 300, and/or other photographing devices) to photograph a user in the second photography mode in S130.

The image analysis unit 103 may analyze the image of a user captured in the second photography mode on the basis of artificial intelligence in S140. In other words, the image analysis unit 103 may analyze or determine the skin type and skin characteristics of the user on the basis of the image of the user captured in the second photography mode.

The disease analysis unit 104 may analyze the image of a user captured in the first photography mode on the basis of artificial intelligence in S140. In other words, the disease analysis unit 104 may detect the wrinkles of the user on the basis of the image of the user captured in the first photography mode, and determine or predict the current disease and/or potential future disease of the user on the basis of the detected wrinkles of the user.

The interface control unit 101 may output result information (e.g., skin type, skin characteristics, and/or disease information) analyzed on the basis of artificial intelligence through the user terminal 200 (e.g., the display of the user terminal 200) or the kiosk 300 (e.g., the display of the kiosk 300) in S150.

The solution provision unit 105 may provide or display information on skin care and/or disease management on the basis of the photographing objective entered by a user through the user terminal 200 (e.g., the display of the user terminal 200) or the kiosk 300 (e.g., the display of the kiosk 300) in S160. For example, the solution provision unit 105 may display a detailed result about the analyzed skin characteristics and/or skin type of a user through the user terminal 200 or the kiosk 300, and may determine a customized skin specialist (e.g., a dermatologist) on the basis of a detailed result about the skin characteristics, skin type, and/or skin trouble of a user, and provide an interface that can connect the user with the determined skin specialist. For example, on the basis of the skin analysis result of a user, the solution provision unit 105 may determine a dermatologist with extensive experience in diagnosing and/or treating blackheads when the user has severe blackheads, or, may determine a dermatologist with extensive experience in diagnosing and/or treating dry skin when the skin of a user is dry and inelastic. The user may have skin problems quickly and easily diagnosed and managed anytime and anywhere by the dermatologist, and may have access to remote consultations and appointment scheduling.

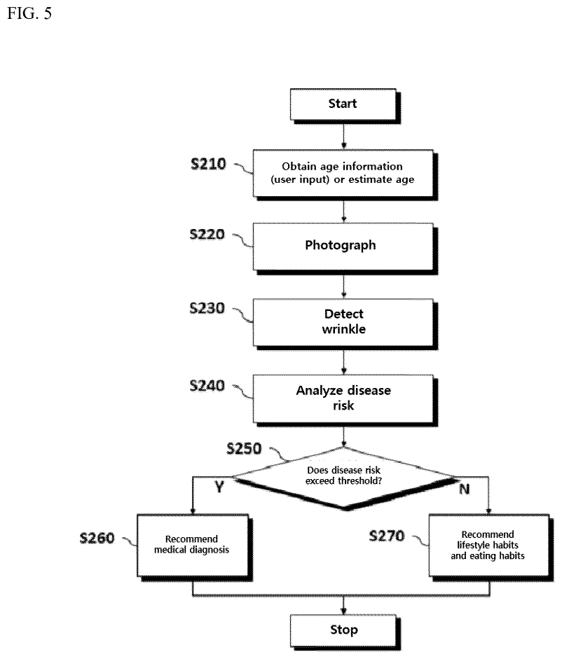

FIG. 5 is a flowchart for analyzing the disease risk of a user on the basis of age information of the user and wrinkle information of the user.

When the interface control unit 101 obtains an input signal indicating that the photographing objective is disease analysis, the interface control unit 101 may provide an interface for receiving the age information of a user through the user terminal 200 or the kiosk 300. The interface control unit 101 may obtain an input signal indicating the age information of a user from the user in S210. In other words, the user may directly input his/her age through the interface provided by the interface control unit 101, and the input age may be transmitted to the interface control unit 101.

The photography control unit 102 may control the photography device (e.g., the camera of the user terminal 200 and/or the camera of the kiosk 300) to photograph a user in the first photography mode in S220. The image analysis unit 103 may detect the wrinkles of a user on the basis of the captured image of the user in S230.

The disease analysis unit 104 may calculate the disease risk in S240. The disease analysis unit 104 may determine the wrinkle occurrence region of a user and calculate the degree of wrinkle occurrence of the user in the region. The disease analysis unit 104 may calculate the disease risk on the basis of the age of a user, the wrinkle occurrence region, and the degree of wrinkle occurrence. The age of a user may be estimated automatically by using age information obtained by entering by a user through the interface control unit 101, or by using an age estimation function based on the facial image of a user.

For example, the disease analysis unit 104 may train an artificial neural network-based age estimation model to estimate the age of a user. The disease analysis unit 104 may input the facial image of a user as input data to an artificial neural network. The disease analysis unit 104 may output the age of a user through the artificial neural network (ANN). The artificial neural network may be implemented by using Single Shot Detector (SSD), Region with CNN (R-CNN), or You Only Look Once (YOLO), which is an object identification algorithm based on a convolutional neural network (CNN).

In an embodiment, the disease analysis unit 104 may perform supervised learning of an age estimation model based on the facial image of a user, and the age estimation model may be implemented as an artificial neural network. The artificial neural network is a predictive model implemented in software or hardware that mimics the computational capabilities of biological systems by using a large number of artificial neurons (or nodes).

The age estimation model based on an artificial neural network may be trained through supervised learning by an age estimation model learning unit by using “a facial image” and “data labeled for age.” In this case, the supervised learning refers to learning that uses data with an input value and an output value corresponding thereto as learning data to find an output value according to a given input value, and refers to learning that is performed when the correct answer is known. For example, labeled data may be data that labels the age of a user. A set of an input value and an output value given to supervised learning is called training data. That is, the above-described “facial image of a user” and “data labeled with age”, which are an input value and an output value, respectively, may be used as training data for supervised learning of the age estimation model.

The disease analysis unit 104 may convert the facial image into an input image of a preset size and input the input image into the age estimation model that has been supervised learned in advance. The disease analysis unit 104 may estimate the age of a user on the basis of the output of the age estimation model. However, it is evident that in estimating the age of a user, not only the age estimation model described above, but also age estimation software or systems that can be utilized at the level of a general technician may be used.

When the calculated disease risk exceeds the first threshold, the disease analysis unit 104 may determine that a disease corresponding to the wrinkle occurrence region has occurred or will occur soon. The disease analysis unit 104 may determine whether the disease risk exceeds the second threshold in S250.

When the calculated disease risk exceeds the preset second threshold that is higher than the first threshold, the solution provision unit 105 may recommend a medical diagnosis to a user in S260. In other words, when the calculated disease risk exceeds the second threshold, the solution provision unit 105 may determine a customized specialist (e.g., a cardiovascular specialist) for the disease corresponding to the wrinkle occurrence region, and provide an interface that can connect the user with the determined cardiovascular specialist. The user may have the disease quickly and easily diagnosed and managed anytime and anywhere by the customized specialist, and may have access to remote consultations and appointment scheduling.

When the disease risk exceeds the first threshold but is lower than or equal to the second threshold, the solution provision unit 105 may display information on recommended lifestyle habits and/or information on recommended eating habits on the user terminal 200 or the kiosk 300 to provide the information to the user in S270.

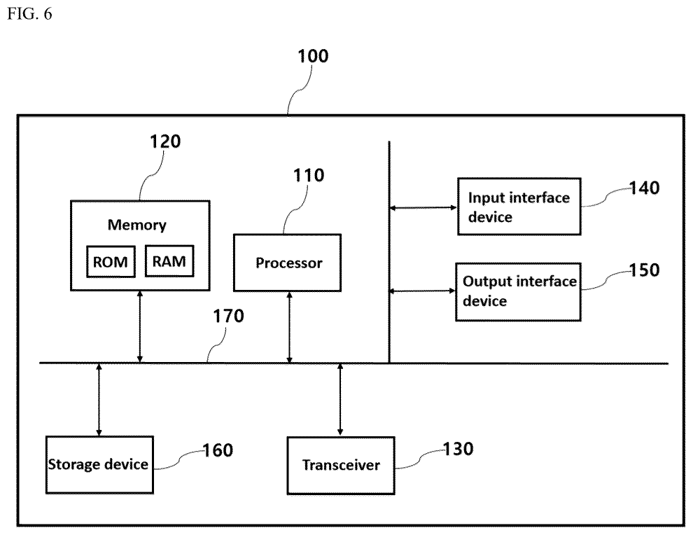

FIG. 6 is a diagram illustrating the configuration of the hardware of the wrinkle-based disease diagnosis server 100 according to FIG. 1.

Referring to FIG. 6, the wrinkle-based disease diagnosis server 100 may include at least one processor 110 and a memory that stores instructions directing the at least one processor 110 to perform at least one operation.

The at least one operation may include at least some of the operations or functions of the wrinkle-based disease diagnosis server 100 described above and may be implemented in the form of instructions and performed by the processor 110.

Here, the at least one processor 110 may refer to a central processing unit (CPU), a graphics processing unit (GPU), or a dedicated processor on which methods according to the embodiments of the present disclosure are performed. Each of a memory 120 and a storage device 160 may be configured as at least one of a volatile storage medium and a non-volatile storage medium. For example, the memory 120 may be one of a read only memory (ROM) and a random access memory (RAM), and the storage device 160 may be a flash memory, a hard disk drive (HDD), a solid state drive (SSD), or various memory cards (e.g., a micro SD card), etc.

Additionally, the wrinkle-based disease diagnosis server 100 may include a transceiver 130 that performs communication via a wireless network. In addition, the wrinkle-based disease diagnosis server 100 may further include an input interface device 140, an output interface device 150, the storage device 160, etc. Each component included in the wrinkle-based disease diagnosis server 100 may be connected to each other by a bus 170 to communicate with each other. FIG. 6 illustrates the wrinkle-based disease diagnosis server 100 as an example, but is not limited thereto. For example, a plurality of user terminals may include the components of FIG. 6.

The methods according to the present disclosure may be implemented in the form of program instructions that can be executed through various computer means and may be recorded in a computer-readable medium. The computer-readable medium may include program instructions, data files, data structures, etc., alone or in combination. The program instructions recorded in the computer-readable medium may be specially designed and configured for the present disclosure or may be known and available to those skilled in the art of computer software.

Examples of computer-readable media may include a hardware device specifically configured to store and execute program instructions, such as ROM, RAM, flash memory, and the like. Examples of the program instructions may include high-level language codes that can be executed by a computer by using an interpreter, etc. as well as machine language codes, such as those produced by a compiler. The hardware device described above may be configured to operate with at least one software module to perform the operations of the present disclosure, and vice versa.

In addition, the above-described method or device may be implemented by combining all or part of configuration or function thereof, or may be implemented by separating them.

Although the present disclosure has been described above with reference to preferred embodiments thereof, it will be understood by those skilled in the art that various modifications and changes may be made to the present disclosure without departing from the spirit and scope of the present disclosure as set forth in the claims below.

1. A server for diagnosing a disease of a user through wrinkle detection, the server comprising:
an interface control unit which displays an interface for selecting a photographing objective through a display linked with the server, and which acquires, from the user, an input signal indicating the photographing objective;
a photography control unit, which controls a skin photography device so as to capture an image of the skin of the user;
an image analysis unit for acquiring the image of the skin of the user from the skin photography device, performing preprocessing on the basis of landmarks based on the image of the skin, and determining a skin type and a skin characteristic of the user on the basis of the preprocessed image of the skin;
a disease analysis unit for detecting wrinkles of the user on the basis of the image of the skin, and diagnosing a disease of the user on the basis of the detected wrinkles of the user; and
a solution provision unit for providing a customized solution according to the diagnosed disease of the user.

2. The server of claim 1, wherein the photography control unit photographs a user in a first photography mode through the skin photography device when obtaining an input signal indicating disease analysis, and photographs the user in a second photography mode through the skin photography device when obtaining an input signal indicating skin analysis, and
the disease analysis unit comprises a wrinkle detection model that is trained by using training data consisting of a training input value corresponding to a skin image of each of multiple users obtained from multiple user terminals, and a training output value corresponding to a wrinkle image or a wrinkle probability map of the user, and generates the wrinkle probability map corresponding to the user on the basis of a deep learning network consisting of a plurality of hidden layers,
inputs the preprocessed skin image of the user into the wrinkle detection model based on a convolutional neural network (CNN), and generates a wrinkle image or a wrinkle probability map corresponding to the skin image on the basis of an output of the wrinkle detection model, and
detects wrinkles of the user on the basis of the wrinkle image or the wrinkle probability map, which is generated.

3. The server of claim 2, wherein the disease analysis unit determines a wrinkle occurrence region of a user, and determines whether the user has a disease on the basis of the wrinkle occurrence region and a degree of wrinkles occurring in the region.

4. The server of claim 3, wherein the disease analysis unit calculates a disease risk on the basis of age of the user, the wrinkle occurrence region, and the degree of the wrinkle, and
sets a weight for the degree of the wrinkle to be low as the age of the user increases, and sets a weight for the degree of the wrinkle to be high as the age decreases on the basis of the obtained age information.

5. The server of claim 4, wherein the solution provision unit provides a medical diagnosis service or information on recommended lifestyle habits and recommended eating habits on the basis of the disease risk.
